SEQUENCE LISTING (11 sequences)

SEQ ID NO: 1 - DNA, 351 bp, Mus musculus
gagcagtgtggcaccatcatgcatggcaatgctgtcaccttctgtgagccgtacggccctcgagagctgaccaccacatgcctgaacacaacaacagcatctgtcctccagttttccattgggtcaggatcatgtcgatttagttactctgaccccagcatcactgtgtcatacgccaagaacaataccgctgattggattcagctggagaaaattagagccccttccaatgtgagcacagtcatccacatcctgtacctccccgaggaagccaaaggggagagcgtgcagttccagtggaaacaggacagcctgcgagtgggtgaggtgtatgaggcctgctgggccctg

SEQ ID NO: 2 - protein, 117 aa, Mus musculus
EQCGTIMHGNAVTFCEPYGPRELTTTCLNTTTASVLQFSIGSGSCRFSYSDPSITVSYAKNNTADWIQLEKIRAPSNVSTVIHILYLPEEAKGESVQFQWKQDSLRVGEVYEACWAL

SEQ ID NO: 3 - DNA, 20 bp, Artificial Sequence
  Description of Artificial Sequence synthetic primer for PCR
atggagcgcggctgctgggc

SEQ ID NO: 4 - DNA, 19 bp, Artificial Sequence
  Description of Artificial Sequence synthetic primer for PCR
aggaacaacaggaacacag

SEQ ID NO: 5 - DNA, 22 bp, Artificial Sequence
  Description of Artificial Sequence synthetic primer for PCR
cctctctccatctttgaggaac

SEQ ID NO: 6 - DNA, 22 bp, Artificial Sequence
  Description of Artificial Sequence synthetic primer for PCR
cagggcccagcaggcctcatac

SEQ ID NO: 7 - DNA, 22 bp, Artificial Sequence
  Description of Artificial Sequence synthetic primer for PCR
ctctcccatctcacagttgctg

SEQ ID NO: 8 - DNA, 21 bp, Artificial Sequence
  Description of Artificial Sequence synthetic primer for PCR
gtaagcagtggcctctgtggg

SEQ ID NO: 9 - DNA, 22 bp, Artificial Sequence
  Description of Artificial Sequence synthetic primer for PCR
tactcgcaccttgctgaaatac

SEQ ID NO: 10 - DNA, 21 bp, Artificial Sequence
  Description of Artificial Sequence synthetic primer for PCR
gagcagtgtggcaccatcatg

SEQ ID NO: 11 - DNA, 18 bp, Artificial Sequence
  Description of Artificial Sequence synthetic primer for PCR
gagcagtgtggcaccatc

(19) United States
(12) Patent Application Publication
(10) Pub. No.: US 2002/0137095 A1
(43) Pub. Date: Sep. 26, 2002

(54) Reelin protein CR-50 epitope region

(75) Inventors: Katsuhiko Mikoshiba, Tokyo (JP); Naoko Tate, Tokyo (JP)

Correspondence Address: FINNEGAN, HENDERSON, FARABOW, GARRETT &, DUNNER LLP, 1300 I STREET, NW, WASHINGTON, DC, 20005, US

(73) Assignee: RIKEN

(21) Appl. No.: 09/897,438

(22) Filed: Jul. 3, 2001

(30) Foreign Application Priority Data
Jul. 4, 2000 (JP) 2000-202801

(51) Int. Cl. C12Q 1/68; G01N 33/53; C07H 21/04; C12N 9/00; C12P 21/02; C12N 5/06; C07K 16/40

(52) U.S. Cl. 435/7.1; 435/69.1; 435/325; 435/320.1; 435/183; 435/6; 536/23.2; 530/388.1

(57) ABSTRACT

Reelin protein itself is further studied for its functions in vertebrates to achieve appropriate diagnosis and treatment of brain disorders resulting from an abnormal reelin gene and abnormally positioned neurons. The present invention provides an epitope region polypeptide of Reelin protein, which comprises a CR-50 antibody recognition site of Reelin protein, but comprises neither a F-spondin domain nor a repeat site; as well as a polynucleotide encoding the polypeptide.

### FIELD OF THE INVENTION

The present invention relates to a CR-50 epitope region polypeptide of Reelin protein and a polynucleotide encoding the polypeptide.

### BACK GROUND OF THE INVENTION

In the mammalian central nervous system (CNS), various classes of neurons are known to migrate from their site of origin to their final positions, where they are arranged in elaborate laminar structures (Rakic, P., (1995) Proc. Natl. Acad. Sci. USA, 92, 11323-11327; Pearlman, A. L. et al., (1998) Curr. Opin. Neurobiol., 8, 45-54; Rice, D. S. & Curran, T., (1999) Genes Dev., 13, 2758-2773). Neocortical development starts from the preplate formation. The preplate lies near the surface of the cortex and is composed of a superficial plexus of corticopetal nerve fibers and earliest-generated neurons, including the Cajal-Retzius and prospective subplate neurons. Consecutively, the preplate is split by the cortical plate neurons into a superficial marginal zone, where the Cajal-Retzius neurons differentiate, and a deep subplate, where the subplate neurons differentiate (Allendoerfer, K. L. & Shatz, C. J. (1994) Annu. Rev. Neurosci., 17, 185-218). The cortical plate neurons are born in the ventricular zone and migrate across the intermediate zone and subplate along radial glial fibers before reaching the cortical plate. The systematic migration of the later-generated neurons past those generated earlier results in an “inside-out” progression in the mammalian cortical plate (Angevine, J. B. & Sidman, R. L. (1961) Nature, 25, 766-768; Rakic, P. (1972) J. Comp. Neurol., 145, 61-84).

A reeler is an autosomal recessive mouse mutant, in which neurons are generated normally but are abnormally positioned, resulting in disorganization of cortical laminar layers in the CNS (Falconer, D. S., (1951) J. Genet., 50, 192-201; Rakic, P. & Caviness, V. S. J., (1995) Neuron, 14, 1101-1104; Stanfield, B. B. & Cowan, W. M. (1979) J. Comp. Neurol. 185, 423-459; Caviness, V. S. Jr. (1982) Dev. Brain Res., 4, 293-302; Caviness, V. S. Jr. & Sidman, R. L., (1973) J. Comp. Neurol., 148, 141-151; deRouvroit, C. L. & Goffinet, A. M. (1998) Adv. Anat. Embryol. Cell Biol., 150, 1-106). In the reeler neocortex, for example, cortical plate neurons are aligned in a practically inverted fashion (“outside-in”). We previously obtained a monoclonal alloantibody, CR-50, by immunizing reeler mice with homogenates of normal embryonic brains (Ogawa, M. et al., (1995) Neuron, 14, 899-912). This antibody was shown to react specifically with Cajal-Retzius neurons in the marginal zone of normal mice. CR-50 antibody was then shown to recognize the Reelin protein itself, which is encoded by the reeler gene (reelin, Reln). Reelin is a secreted extracellular matrix protein composed of 3,461 amino acids with a relative molecular mass of 388 kDa (D'Arcangelo, G. et al., (1997) J. Neurosci. 17, 23-31; D'Arcangelo, G. et al., (1995) Nature, 374, 719-723) (FIG. 1A). The N-terminus of Reelin has 25% identity with that of F-spondin, an extracellular matrix protein that controls the adhesion and extension of commissural axons in the spinal cord (Klar, A. et al., (1992) Cell 69, 95-110). The first 500 amino acids of Reelin are followed by eight “Reelin repeats,” each of which is composed of 350-390 amino acids and contains an EGF-like motif of 30 amino acids in the middle (D'Arcangelo, G. et al., (1995) Nature, 374, 719-723). The highly charged C-terminus of Reelin is essential for its secretion into the extracellular space (D'Arcangelo, G. et al., (1997) J. Neurosci. 17, 23-31; deBerbeyck, V. et al., (1997) Mol. Brain Res., 50, 85-90).

In the cytoplasm, the Reelin signal is received by the cytosolic adapter protein, Disabled homolog 1 (Dab1) (Howell, B. W. et al., (1997) EMBO J. 16, 121-132; Howell, B. W. et al., (1997) Nature 389, 733-737; Howell, B. W. et al., (1999) Genes Dev. 13, 643-648; Sheldon, M. et al., (1997) Nature, 389, 730-733; Ware, M. L. et al., (1997) Neuron, 19, 239-249; Rice, D. S. et al., (1998) Development, 125, 3719-3729), whose defect causes reeler-like phenotype in mice (Howell, B. W. et al., (1997) Nature 389, 733-737; Sweet, H. O. et al., (1996) Mamm. Genome, 7, 798-802, J. Biol. Chem., 273, 556-3560; Yoneshima, H. et al., (1997) Neurosci. Res., 29, 217-223). Dab1 binds to non-phosphorylated NPxY motifs in the cytoplasmic domains of several cell surface proteins (Pawson, T. & Scott, J. D. (1997) Science, 278, 2075-2080; Trommsdorff, M. et al., (1998) J. Biol. Chem., 273, 3556-3560; Homayouni, R. et al., (1999) J. Neurosci., 19, 7507-7515; Howell, B. et al., (1999) Mol. Cel. Biol., 19, 5179-5188). Reelin induces tyrosine phosphorylation of Dab1 (Howell, B. W. et al., (1999) Genes Dev. 13, 643-648). It was also shown that the phenotype of double-knockout mice lacking both the very low density lipoprotein receptor (VLDLR) gene and the apolipoprotein E receptor 2 (ApoER2) gene is indistinguishable from that of reeler mice (Trommsdorff, M. et al., (1999) Cell, 97, 698-701). Recently, Reelin has been demonstrated to bind to VLDLR, ApoER 2 (D'Arcangelo, G. et al., (1999) Neuron, 24, 471-479; Hiesberger, T. et al., (1999) Neuron, 24, 481-489), and cadherin-related neuronal receptor (CNR) family members (Senzaki, K. et al., (1999) Cell 99, 635-647), leading to Dab1 tyrosine phosphorylation. These results suggest that Reelin controls neuronal migration and positioning by stimulating tyrosine phosphorylation of Dab1 via VLDLR, ApoER2, and CNR family proteins.

CR-50 antibody has neutralizing activity of Reelin functions both in vitro and in vivo (Ogawa, M. et al., (1995) Neuron, 14, 899-912; DelRio, J. Q. et al., (1997) Nature, 385, 70-74; Miyata, T. et al., (1997) J. Neurosci., 17, 3599-3609; Nakajima, K. et al., (1997) Proc. Natl. Acad. Sci. USA 94, 8196-8201). Recently, we reported that an epitope against the CR-50 antibody is located near the N-tenninus of Reelin (D'Arcangelo, G. et al., (1997) J. Neurosci. 17, 23-31). Because CR-50 antibody inhibits Reelin functions, this CR-50 epitope region should play a crucial role in the Reelin signaling during neurodevelopment. Accordingly, Reelin protein itself should be further studied regarding its functions in vertebrates to achieve diagnosis and treatment suitable for brain disorders resulting from an abnormal reelin gene and abnormally positioned neurons. For this purpose, it is necessary to identify the CR-50 epitope region of Reelin and to examine its functions in Reelin protein.

### SUMMARY OF THE INVENTION

We found that Reelin proteins bind to each other to form a large protein complex both in vitro and in vivo. Interestingly, this assembly formation is clearly inhibited by CR-50 antibody at a concentration known to neutralize Reelin functions.

Although we previously found a truncated Reelin protein in Xenopus, which contains a F-spondin domain, a CR-50 recognition site and no repeat site (Japanese Patent Application No. 2000-109954), we performed a further study in view of the above findings, thereby providing an isolated polypeptide of the most important epitope region for binding with CR-50 and for the Reelin protein complex formation newly discovered in the present invention. We also found the utility of this polypeptide ahead of others.

Namely, we found that the CR-50 epitope region corresponds to amino acids 230-346 of Reelin protein (SEQ ID NO: 2). When a polynucleotide encoding this epitope region (SEQ ID NO: 1) was introduced into an expression vector and then expressed in E. coli cells to produce a recombinant polypeptide of CR-50 epitope region for a further study, we also found that such recombinant epitope polypeptides spontaneously form a regular homopolymer via electrostatic interaction, and that this homopolymer formation closely correlates with the assembly formation and functions of the full-length Reelin protein, thereby finally completing the present invention.

To summarize, the present invention provides the following embodiments (1) to (11).

(1) A CR-50 epitope region polypeptide of Reelin protein, which comprises a CR-50 antibody recognition site of Reelin protein, but comprises neither a F-spondin domain nor a repeat site.

As used herein, the term “Reelin protein” or “Reelin” means a protein encoded by a causative gene of the reeler mutant mouse. This protein is an extracellular matrix protein containing a signal peptide, a F-spondin domain, a CR-50 recognition site and Reelin repeats.

The term “F-spondin domain” means an N-terminal region of the Reelin protein that has homology to F-spondin.

The term “CR-50 recognition site” means a site recognized by CR-50 antibody in the case of mouse Reelin proteins, or a site homologous to the mouse Reelin CR-50 recognition site in the case of other organisms' Reelin proteins. In addition, CR-50 antibody may be produced as described in Neuron 14, 899-912 (1995).

The term “repeat site” means a region of the Reelin protein that contains sequentially repeated amino acid sequence units homologous to one another, each having an EGF-like motif in the middle.

(2) The CR-50 epitope region polypeptide of Reelin protein according to (1) above, which is derived from a mouse.

(3) A polypeptide selected from the group consisting of:

(a) a polypeptide comprising the amino acid sequence shown in SEQ ID NO: 2; and

(b) a polypeptide capable of binding to CR-50 antibody, which comprises deletion, substitution or addition of one or more amino acids in the amino acid sequence shown in SEQ ID NO: 2.

(4) A polynucleotide encoding the polypeptide according to any one of (1) to (3) above.

(5) A polynucleotide selected from the group consisting of:

(a) a polynucleotide comprising the nucleotide sequence shown in SEQ ID NO: 1;

(b) a polynucleotide encoding a polypeptide capable of binding to CR-50 antibody, which comprises deletion, substitution or addition of one or more nucleotides in the nucleotide sequence shown in SEQ ID NO: 1; and

(c) a polynucleotide comprising a degenerate nucleotide sequence of polynucleotide (a) or (b).

The expression “one or more” found in (3) and (5) above means the number of mutations which can be induced by site-specific mutagenesis or the like without impairment of functions through mutation.

The term “degenerate” means that two or more polynucleotides, each having a different nucleotide sequence, encode the same amino acid or amino acid sequence due to degeneracy of the genetic code.

(6) An expression vector comprising the polynucleotide according to (4) or (5) above.

A vector which can carry the above polynucleotide includes, but is not limited to, those known to be available in the art and suitable for expression of a polypeptide encoded by the above polynucleotide, for example, pET-29a (Novagen) and pcDNA3 (Invitrogen).

As known in the art, it is also possible to incorporate into an expression vector a regulatory sequence which may control expression of the above polynucleotide, such as promoter and/or enhancer, as needed.

Further, the polypeptide of the present invention may be expressed alone, or expressed as a fusion protein with other protein such as glutathione-S-transferase (GST) or with a tag such as histidine-tag, according to purposes.

(7) A host cell transfected with the expression vector according to (6) above.

Any host cell capable of expressing the above polypeptide may be used. Illustrative examples include animal, plant and bacterial cells known to be available in the art, with E. coli cells, 293T cells and COS cells being preferred.

(8) A method for producing the polypeptide according to any one of (1) to (3) above, which comprises culturing the host cell according to (7) above.

(9) A method for stimulating the assembly of Reelin protein molecules, which comprises adding the polypeptide according to any one of (1) to (3) above or the polynucleotide according to (4) or (5) above.

(10) A composition for stimulating the assembly of Reelin protein molecules, which comprises the polypeptide according to any one of (1) to (3) above or the polynucleotide according to (4) or (5) above.

(11) A pharmaceutical composition for diagnosis and/or treatment of diseases resulting from abnormally positioned neurons, which comprises the polypeptide according to any one of (1) to (3) above or the polynucleotide according to (4) or (5) above.

This specification includes part or all of the contents as disclosed in the specification and/or drawings of Japanese Patent Application No. 2000-202801, which is a priority document of the present application.

### BRIEF DESCRIPTION OF THE DRAWINGS

FIG. 1 shows that native Reelin molecules form a large protein complex. (A) Schematic representation of the Reelin's primary structure. CR-50 antibody recognizes upstream of Reelin repeats. (B-D) Immunoblot with anti-Reelin antibody. (B) Homogenates of reeler heterozygote (rl/+) and homozygote (rl/rl) cerebra (E18) were run through SDS/PAGE. (C) Homogenates of cerebra (E18) and cerebella (P5) of reeler heterozygotes and homozygotes were separated by native PAGE. (D) Supernatants of cerebellar primary cultures of reeler heterozygote and homozygote (P5) were loaded onto native PAGE gels.

FIG. 2 shows cell adhesion assay indicating that Reelin molecules bind to each other. (A & B) Immunostaining of reelin-transfected 293T cells with CR-50 antibody. When CR-50 was added to the living cells, punctate staining on the cell surface was observed (A), whereas cytoplasm was stained diffusely when CR-50 was added after the cells were fixed and permeablized (B). (C-F) Cell adhesion assay. (C) Reelin-presenting cells on Reelin-coated dish. (D) Reelin-presenting cells on control-coated dish. (E) Control cells on Reelin-coated dish. (F) Control cells on control-coated dish. (G) Histograms showing cell numbers that are normalized to (F), which was scored as 100. Numbers are the mean value of five independent experiments±standard deviation (SD).

FIG. 3 shows that CR-50 antibody inhibits homophilic interaction of Reelin molecules. (A-E) Inhibition of cell adhesion by CR-50 antibody. (A) Reelin-presenting cells on Reelin-coated dish. (B-D) Reelin-presenting cells on Reelin-coated dish after incubation with 20, 50 and 200 μg/ml CR-50 antibody, respectively. (E) Histograms showing cell numbers of each case (A-D) that are normalized to (A). Numbers are the mean value of five independent experiments±SD. (F) Inhibition of Reelin assembly by CR-50 antibody. reeler heterozygote cerebellar (P5) cells were cultured in the presence of no mouse IgG (−), non-immunized mouse IgG, or CR-50 antibody. The supernatants of these cultures were separated by native PAGE and blotted with anti-Reelin.

FIG. 4 shows that Reelin binds to the CR-50 epitope region in cell adhesion assay. (A) Reelin-presenting cells on CR-50 epitope-coated dish (200 μg/ml). (B) Reelin-presenting cells on control-coated dish. (C) Histograms showing cell numbers in cell adhesion assay on CR-50 epitope-coated dishes (200 or 50 μg/ml) that are normalized to the control.

FIG. 5 shows that CR-50 epitope fragments form a homopolymer. (A) Elution profile of gel filtration chromatography showing that CR-50 epitope fragments form a homopolymer with a molecular mass of more than 600 kDa at 0.15 M NaCl. (B-D) The polymer is dissociated at a higher ionic strength [0.5 M NaCl (B); 0.75 M NaCl (C); 1.0 M NaCl (D)]. The polymer peak appears at an elution time of 7.7 min and a monomer peak at 15.2 min. V₀ indicates the void volume of column.

FIG. 6 shows structural analysis of CR-50 epitope polymer. (A) Absorbance spectra of Congo red with CR-50 epitope polymer (a) and without CR-50 epitope polymer (1)) were observed. A red-shift in λₘₐₓ occurred when CR-50 epitope polymer was added. (B) The spectral difference between the polymer-containing solution and a dye-only solution was detected. (C) CD spectra of CR-50 epitope fragments at various NaCl concentrations were observed. (D-F) Electron micrographs of CR-50 epitope polymer by rotary shadowing method (D and E) or negative staining method (F). (Bar=100 nm)

### DETAILED DESCRIPTION OF THE INVENTION

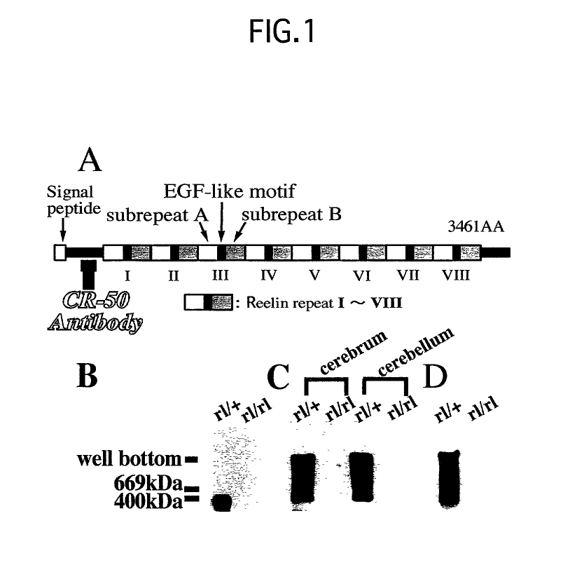

When living embryonic brain cells are stained with anti-Reelin antibodies, the cell surface of Reelin-secreting cells, such as Cajal-Retzius neurons in the neocortex, are well labeled in punctate pattern (Ogawa, M. et al., (1995) Neuron, 14, 899-912; deBerbeyck, V. et al., (1997) Mol. Brain Res., 50, 85-90; Miyata, T. et al., (1997) J. Neurosci., 17, 3599-3609; Nakajima, K. et al., (1997) Proc. Natl. Acad. Sci. USA, 94, 8196-8201). These data suggest that Reelin protein molecules are assembled and anchored to the surface of the Reelin-secreting cell membrane. To determine whether the Reelin protein molecules indeed assemble in vivo, Western blot analysis was performed after electrophoresis on both non-denaturing and denaturing gels. When embryonic cerebral homogenates [embryonic day 18 (E18)] were separated under the denaturing conditions, immunoblotting with an anti-Reelin antibody revealed a band of 400 kDa in heterozygous reeler (Reln rl/+; rl/+) (FIG. 1B). This apparently corresponds to a monomer of the Reelin protein. In contrast, when cerebral (E18) and cerebellar [postnatal day 5 (P5)] homogenates were run through non-denaturing native gels, a broad range of bands spread from 400 kDa to the well were detected in addition to the monomer band in heterozygotes (rl/+) (FIG. 1C).

The status of Reelin secreted from cultured cerebellar granule cells was then investigated. The supernatants of the primary cerebellar cultures of reeler homozygotes (rl/rl) and heterozygotes were resolved on non-denaturing native gel and probed with anti-Reelin antibody. Again, only the samples from heterozygous mice showed a wide spread of bands with molecular masses higher than 400 kDa (FIG. 1D). All these results taken together suggested that secreted Reelin is in a large protein complex state both in vitro and in vivo.

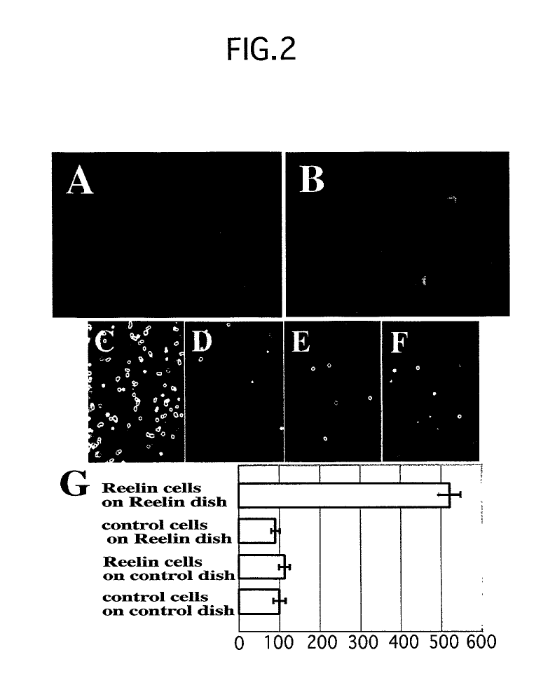

A cell adhesion assay was then performed to examine whether Reelin protein molecules indeed show homophilic interaction. Some of the secreted Reelin is found to be anchored to the surface of Reelin-producing brain cells (Ogawa, M. et al., (1995) Neuron, 14, 899-912; deBerbeyck, V. et al., (1997) Mol. Brain Res., 50, 85-90; Miyata, T. et al., (1997) J. Neurosci., 17, 3599-3609; Nakajima, K. et al., (1997) Proc. Natl. Acad. Sci. USA, 94, 8196-8201). Thus, living reelin-transfected 293T cells were stained with CR-50 antibody to see whether the secreted Reelin is anchored to the 293T cells as well (FIG. 2A). The surface of the living 293T cells was stained in a punctate pattern similar to that of living brain cells, whereas fixed cells were stained diffusely in the cytoplasm (FIG. 2B). These results indicate that some of the secreted Reelin molecules are anchored to the surface of the transfected 293T cells.

When reelin-transfected 293T cells were plated onto Reelin-coated dishes, much more cells bound to them (FIG. 2C) than to control dishes, which had been coated with supernatants containing no Reelin (FIGS. 2D and 2G). Control cells transfected only with a backbone vector did not bind to Reelin-coated or control dishes (FIGS. 2E-2G). Thus, it is highly likely that Reelin molecules have homophilic interaction. Indeed, when an expression cDNA library with a fragment of Reelin was screened to search molecules interacting with it, the population of reelin cDNA itself was found to be increasingly enriched by the second and third screening (data not shown). Taken together, these results show that Reelin protein molecules bind to each other to form a large protein complex.

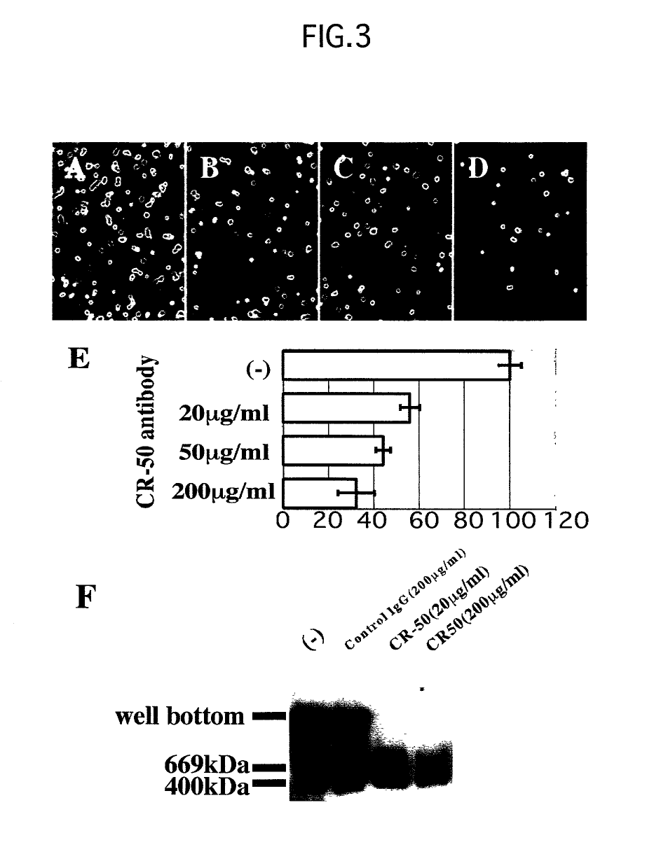

To further investigate the relationship between this homophilic interaction of Reelin molecules and Reelin functions, CR-50 antibody was examined for its effect on the assembly formation of Reelin molecules (FIG. 3). The assembly formation of Reelin molecules was inhibited by the addition of CR-50 antibody, indicating that this assembly formation is related to physiological functions of Reelin protein and induced by an epitope region itself. To confirm this, only an epitope region, which is a recognition site for CR-50 antibody, was expressed.

An epitope region of Reelin protein was identified as follows. First, gene sequences partially encoding various parts of the reelin gene were integrated to create many gene expression systems systematically. A protein encoded by each gene sequence was produced in an in vitro transcription/translation experiment (according to Promega's protocols). Each protein was analyzed by Western blotting to determine whether it is immunoprecipitated with CR-50 antibody, thereby identifying an epitope region recognized by CR-50 antibody.

The analysis showed that the CR-50 epitope region of Reelin protein is in amino acids 230-346 (SEQ ID NO: 2).

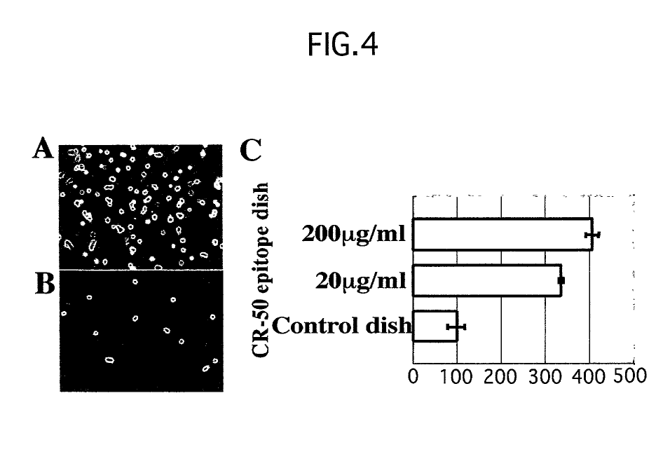

To determine whether the Reelin assembly is mediated by the CR-50 epitope region itself, Petri dishes were coated with purified recombinant CR-50 epitope fragments and a cell adhesion assay was performed by using full-length reelin-transfected 293T cells. When Reelin-presenting cells were plated onto the CR-50 epitope-coated dishes, much more cells bound to them in a dose-dependent manner than to the control dish (FIG. 4). These results indicate that Reelin protein molecules bind to the purified CR-50 epitope fragments, suggesting that the homophilic interaction of Reelin molecules is mediated by the CR-50 epitope region itself.

In view of the foregoing, it is clear that an abnormal Reelin protein in a sample can be identified, for example, by detecting the presence or absence and/or degree of Reelin assembly in the sample. The detection may be carried out in any manner, for example, by extracting Reelin protein or a gene encoding it from a sample; purifying and/or amplifying it, if necessary; and then analyzing it by using one or more techniques selected from the group consisting of electrophoresis, gel filtration chromatography, cell adhesion assay and electron microscopy.

Also, plates coated with the CR-50 epitope region may be prepared and used for screening epitope-binding substances, including Reelin protein, in a sample. When plates are coated with CR-50 antibody instead of the CR-50 epitope region, Reelin protein containing the epitope region can be more specifically screened. In these screening assays, the presence or absence of binding can be detected, for example, by using a secondary antibody conjugated with fluorescent dye, as apparent to those skilled in the art.

Further, it is possible to provide a kit for detecting the presence or absence and/or amount of Reelin protein or its fragment containing the epitope region in a sample, which comprises a plate coated with CR-50 antibody.

Furthermore, the in vivo assembly formation of Reelin protein molecules can be stimulated by adding the polypeptide of the present invention or a polynucleotide encoding it. In this case, the polypeptide of the present invention or a polynucleotide encoding it may be added alone or as a composition for stimulating the Reelin assembly. The composition may also contain any other ingredient generally used in the art, so long as the object of the present invention can be achieved.

Reelin protein was found to require the assembly formation in vivo for its normal functions, but on the other hand, a method for inhibiting the Reelin assembly is also provided in order to clarify Reelin functions. A composition for inhibiting the Reelin assembly may comprise, for example, an antisense oligonucleotide against a polynucleotide encoding the CR-50 epitope region of Reelin protein.

The present invention can also provide a pharmaceutical composition for treatment of diseases resulting from abnormally positioned neurons, which comprises the polypeptide of the present invention or a polynucleotide encoding it. For example, diseases resulting from abnormally positioned neurons, such as lissencephaly, polymicrogyria, and ectopic gray matter, may be treated by integrating a polynucleotide encoding the polypeptide of the present invention into an expression vector, introducing the vector into cells derived from a patient's tissue, such as neuroblasts or neural stem cells, and then transplanting the cells into the patient's brain. The pharmaceutical composition may further contain pharmaceutically acceptable carriers, excipients and/or buffers, which are commonly used for pharmaceutical compositions, in addition to the polypeptide of the present invention or a polynucleotide encoding it.

### EXAMPLES

The present invention will be further described in the following examples. The examples are provided for illustrative purposes only, and are not intended to limit the scope of the invention.

Animal

reeler mice derived from heterozygous B6C3Fe-a/a-rl adults (Jackson Laboratory, Bar Harbor, Me.) were used. Homozygous and heterozygous mice were obtained by mating homozygous males with heterozygous females. The day at which a vaginal plug was detected and the day of birth were designated as E0 and P0, respectively.

Cerebellar Primary Culture

Single-cell suspensions were prepared from cerebella from reeler mice (P5). Cell suspensions in basal Eagle's medium with 10% FCS were plated onto poly-L-lysine-coated plastic dishes at 5×10⁵ cells/cm². The cultures were switched to serum-free medium 18 hours after plating, followed by incubation for 4 days, and the culture supernatants were collected. CR-50 antibody was added to the serum-free medium from the beginning of the primary culture, when its effect on the assembly formation was examined.

Electrophoresis and Western Blot Analysis

SDS-PAGE and native gel electrophoresis of Reelin were performed. After electrophoresis, proteins were transferred onto filters (Millipore). After blocking, the filters were incubated with a primary antibody (G10: anti-Reelin monoclonal antibody; J. Neuroscience Methods, 82, 17-24, 1998) for 3 hours at room temperature. After incubation, the filters were washed and incubated with a peroxidase-conjugated anti-mouse secondary antibody (Jackson ImmunoResearch). After incubation for one hour, the filters were washed, and then visualized using a chemiluminescent detection system (Boehringer-Mannheim). Homogenate protein (100 μg) or the cerebellar culture supernatant (20 μg) was used for this analysis.

Immunohistochemical Staining

293T cells were transfected with the full-length mouse reelin expression construct (pCrl; kindly provided by T. Curran; D'Arcangelo et. al., J. Neuroscience, January 1, 17(1):23-31, 1997). Two days after transfection, cells were fixed with 4% PFA, treated with 0.05% Triton X-100 and then blocked with 10% horse serum. Immunostaining was performed with purified CR-50 antibody at a 1:200 dilution from a 5 mg/ml stock. A secondary antibody was used at a 1:100 dilution (Fluorescein Isothiocyanate (FITC) conjugated anti-mouse IgG). In the case of living cell surface staining, CR-50 was added directly to the medium of cultured cells at a 1:200 dilution and incubated for 30 minutes at 37° C. The cells were then fixed, blocked and incubated with the secondary antibody.

### Example 1

Determination of Reelin CR-50 Epitope Region

Polynucleotides corresponding to the following different parts of the full-length amino acid sequence of Reelin protein were prepared by PCR method:

| | |
|---|---|
| 1-1800 (primers: ATGGAGCGCGGCTGCTGGGC | (SEQ ID NO: 3) and |
| AGGAACAACAGGAACACAG (SEQ ID NO: 4)); | |
| 1-460 (primers: ATGGAGCGCGGCTGCTGGGC | (SEQ ID NO: 3) and |
| CCTCTCTCCATCTTTGAGGAAC (SEQ ID NO: 5)); | |
| 1-346 (primers: ATGGAGCGCGGCTGCTGGGC | (SEQ ID NO: 3) and |
| CAGGGCCCAGCAGGCCTCATAC (SEQ ID NO: 6)); | |
| 1-230 (primers: ATGGAGCGCGGCTGCTGGGC | (SEQ ID NO: 3) and |
| CTCTCCCATCTCACAGTTGCTG (SEQ ID NO: 7)); | |
| 1-190 (primers: ATGGAGCGCGGCTGCTGGGC | (SEQ ID NO: 3) and |
| GTAAGCAGTGGCCTCTGTGGG (SEQ ID NO: 8)); | |
| 190-346 (primers: TACTCGCACCTTGCTGAAATAC | (SEQ ID NO: 9) and |
| CAGGGCCCAGCAGGCCTCATAC (SEQ ID NO: 6)); and | |
| 230-346 (primers: GAGCAGTGTGGCACCATCATG | (SEQ ID NO: 10) and |
| CAGGGCCCAGCAGGCCTCATAC (SEQ ID NO: 6)). | |

Each of these polynucleotides was integrated into pcDNA3 vector (Invitrogen; having Cytomegalovirus (CMV) promoter and T7 promoter) using ligase to construct a gene expression system. DNA encoded by each expression system was purified and 2 μg of the DNA was then reacted with T7 RNA polymerase to synthesize RNA, from which each protein was synthesized using a reticulocyte lysate transcription/translation system (TnT, Promega). Each synthesized protein was subjected to an immunoprecipitation experiment as follows. First, 50 μl of each protein solution was diluted to 1 ml in RIPA buffer (50 mM Tris, pH8.0, 150 mM NaCl, 1% Nonidet P40, 0.5% sodium deoxycolate, 0.1% SDS). Each solution also contained protease inhibitors: leupeptin (20 μ/ml), aprotinin (0.1 mg/ml) and phenylmethylsulfonyl fluoride (2 mM). To each protein solution, 50 μl of ascites fluid containing CR-50 antibody was added and then incubated at 4° C. for 24 hours. The solution was then mixed with Immobilized G-protein agarose beads (Immunopure plus, Pierce), incubated at 4° C. for 30 minutes, and centrifuged to collect the resulting immunoprecipitates, which were then washed with RIPA buffer three times. The immunoprecipitates were re-suspended in 20 μl of SDS-sample buffer and the electrophoresis of them was performed. Based on the fact that a protein containing the CR-50 epitope is immunoprecipitated, the CR-50 epitope region (230-346; SEQ ID NO: 2) was determined from a series of results.

### Example 2

Expression and Purification of Reelin Epitope Protein

The Reelin epitope fragment (residues 230-346; SEQ ID NO: 2) against CR-50 antibody was expressed as a His-tag fusion protein in E. coli cells.

The epitope fragment was cloned into pET-29a (Novagen) by PCR amplification of a polynucleotide encoding the CR-50 epitope region of Reelin (SEQ ID NO: 1) using primers GAGCAGTGTGGCACCATC (SEQ ID NO: 11) and CAGGGCCCAGCAGGCCTCATAC (SEQ ID NO: 6). This insert encoding the CR-50 epitope region was ligated into pET-29a vector which carries a C-terminal His-tag sequence (Novagen) using T4 DNA ligase and transformed into competent BL21/pLysS E. coli cells (Novagen). The sequence of the insert was confirmed by DNA sequencing (ABI PRISM Model 3700). Expression of a recombinant Reelin epitope polypeptide was induced by IPTG, and the CR-50 epitope protein was purified from the E. coli extract according to a procedure based on pET system protocols using a HisBind resin affinity column. In more detail, E. coli cells were collected, re-suspended and sonicated in a buffer containing 5 mM imidazole, 20 mM Tris-HCl (pH 8.0), 0.5 M NaCl and 0.2 mM PMSF. After centrifugation, the supernatant was loaded onto a HisBind resin affinity column (Novagen).

### Example 3

Cell Adhesion Assay

To investigate whether the assembly of Reelin is related to its physiological functions, the effect of the function-blocking anti-Reelin antibody, CR-50 (Ogawa, M. et al., (1995) Neuron, 14, 899-912; DelRio, J. Q. et al., (1997) Nature, 385, 70-74; Miyata, T. et al., (1997) J. Neurosci., 17, 3599-3609), on the homophilic interaction of Reelin molecules was examined.

293T cells transfected with pCrl that contains the full-length reelin open reading frame and those transfected with vector pcDNA3 (Invitrogen) alone were cultured in DMEM+10% fetal calf serum (FCS) for 5 days, and each of the supernatants was collected. The former supernatant contained full-length Reelin molecules and the latter one contained no Reelin. After nitrocellulose/methanol treatment, Petri dishes were coated overnight with each supernatant. Petri dishes coated with the CR-50 epitope were prepared by overnight incubation with recombinant CR-50 epitope proteins produced from E. coli cells. Then, 293T cells, which had been transfected with pCrl or pcDNA3 48 hours before, were plated onto the Reelin-coated dishes or control dishes coated with the culture supernatant of pcDNA3 transfected 293T cells. After incubation for 2 hours, each plate was washed with panning buffer (PBS containing 10% v/v FCS) 7 to 10 times until no floating cells remained. To examine the effect of CR-50 antibody on cell adhesion, the antibody was added to the culture medium from the beginning of incubation. Then, the cells remaining on the dishes were observed by a microscope connected to a computer-controlled camera. This experiment was repeated five times, and for each dish, five microscopic fields were scored.

In the cell adhesion assay, when CR-50 antibody was added to the culture medium, it inhibited adhesion in a dose-dependent manner (FIGS. 3A-3E). At 20, 50 or 200 μg/ml CR-50 antibody, the number of bound cells was decreased to 56%, 44% and 32% of the control, respectively (FIG. 3A). Taking into account that the cell adhesion assay shows about 20% of nonspecific background binding (FIGS. 2D-2G), 20, 50 and 200 μg/ml CR-50 antibody can inhibit the specific binding to approximately one-third, one-fourth and one-tenth, respectively. Because the cell adhesion assay gives high background in general, due to technical difficulty, the effect of CR-50 antibody on the assembly of Reelin that is secreted into the medium was further examined. Cerebellar granule cells were cultured with this antibody, and the supernatant was analyzed by Western blotting. Interestingly, even at 20 μg/ml CR-50 antibody, broad high-molecular-weight bands were not detected at all (FIG. 3F). When non-immunized mouse IgG was added to the culture medium, the homophilic Reelin complexes were formed normally. Thus, CR-50 antibody clearly inhibits the homophilic binding of Reelin molecules even at 20 μg/ml. Because CR-50 antibody shows a function-blocking effect on Reelin from around this concentration (Ogawa, M. et al., (1995) Neuron, 14, 899-912), the inhibitory effect on homophilic interaction correlates well with the blocking activity of Reelin functions. These results suggest that the CR-50 epitope region is a critical region in the assembly of Reelin protein molecules and that the formation of homophilic Reelin complexes may have an essential role in physiological functions of Reelin.

### Example 4

Gel Filtration Chromatography

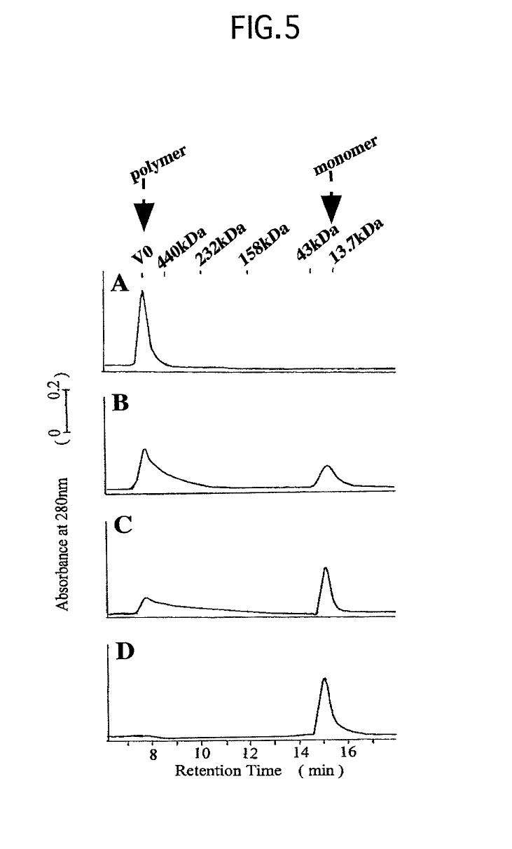

To investigate the structural and physical features of the CR-50 epitope, the CR-50 epitope fragment was analyzed by analytical gel filtration chromatography to see whether the epitope could assemble to form a homopolymer (FIG. 5).

A FPLC gel filtration chromatography system (Pharmacia) was used for chromatography. This system was controlled by a computer FPLC director program (Pharmacia). The sample was loaded onto a Superdex-200 gel filtration column (separation range: 10 kDa to 600 kDa, Pharmacia), which had been pre-equilibrated with a buffer containing 20 mM sodium phosphate, pH 7.4, 0.15 M NaCl, and 5 mM DTT, and then eluted at flow rate of 0.5 ml/min. The sample concentration was adjusted to 40 μM.

Under physiological conditions (0.15 M NaCl, pH 7.4), the elution profile of the CR-50 epitope fragments showed only a polymer peak at the void volume (V₀) of this column (FIG. 5A). This suggests that all of the fragments were associated in a huge complex with a molecular mass larger than 600 kDa. The molecular mass of the CR-50 epitope monomer is about 15 kDa, so this polymer is composed of more than 40 CR-50 epitope molecules. Even in the presence of 10 mM dithiothreitol (DTT), this polymer was detected.

### Example 5

Electron Microscopy

The structure of CR-50 epitope polymer was investigated using electron microscopy. The whole images of CR-50 epitope polymer were observed using the rotary shadowing method and the negative staining method (Katayama, E., (1989) J. Biochem., 106, 751-70). A solution of CR-50 epitope protein was applied onto carbon-coated copper grids. In the rotary shadowing method, the grids were then rotary shadowed with platinum. In the case of negative staining method, the grids were negatively stained with 1% uranyl acetate, washed and air-dried. These samples were examined with a JEOL 2000EX transmission electron microscope (JEOL Ltd.) operated at an acceleration voltage of 80 kV. The concentration of the sample for electron microscopy was adjusted to 0.2 μM.

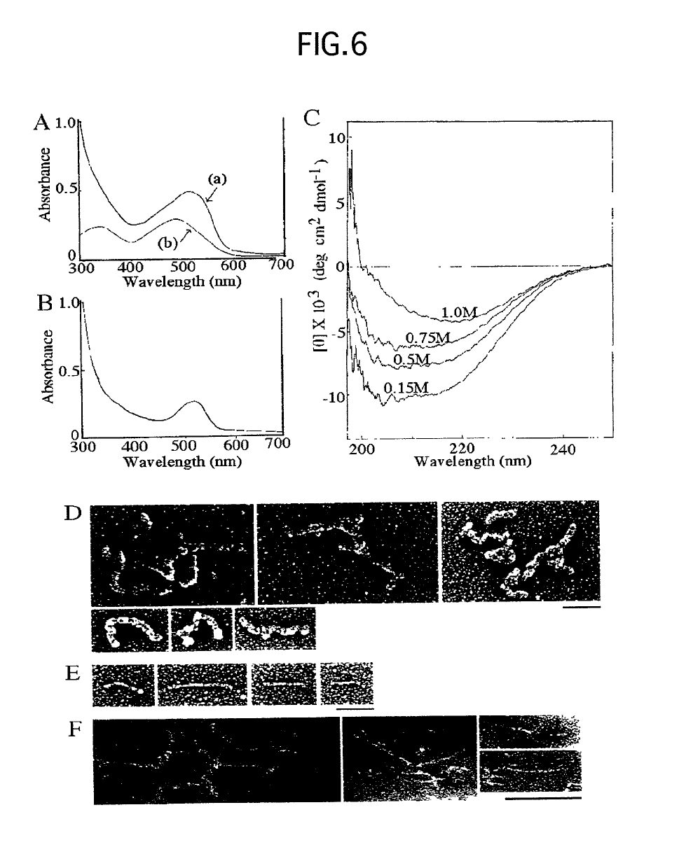

Electron micrographs revealed numerous, string-like polymer structures, 100-400 nm in length regardless of the presence of 10 mM DTT (FIGS. 6D-6F). The polymers shown in FIG. 6D appear to have a twisted structure of finer polymers. In fact, some (less than 3% of the whole population) finer polymers with straight structure were also observed (FIG. 6E).

### Example 6

Congo Red Measurement

A solution of the CR-50 epitope was transparent without any insoluble sediments, which are often experienced by irregular aggregation when using bacterially produced recombinant proteins. To confirm further that the CR-50 epitope polymer is different from the irregular aggregates, samples were tested for Congo red binding by the spectroscopic band-shift assay (Klunk, W. E. et al., (1989) J. Histochem. Cytochem., 37, 1273-1279; Klunk, W. E. et al., (1989) J. Histochem. Cytochem., 37, 1293-1297; Klunk, W. E. et al., (1999) Anal. Biochem., 266, 66-76). Congo red is known to bind amyloid-like fibrils with a β-sheet structure in a regular parallel arrangement, which can be detected by a red-shift in the Congo red absorbance spectrum (Klunk, W. E. et al., (1999) Anal. Biochem., 266, 66-76). Congo red analysis was performed as previously described in Klunk, W. E. et al., (1989) J. Histochem. Cytochem., 37, 1273-1279; Klunk, W. E. et al., (1989) J. Histochem. Cytochem., 37, 1293-1297; and Klunk, W. E. et al., (1999) Anal. Biochem., 266, 66-76. Protein was added to 500 μl of 10 μM Congo red buffer containing 20 mM sodium phosphate and 0.15 M NaCl (pH 7.4). The reaction sample was thoroughly mixed and incubated at room temperature for at least 30 minutes before recording its absorbance spectrum by a Jusco 660 Spectrophotometer.

Addition of the CR-50 epitope polymer to the Congo red solution induced a red-shift in λₘₐₓ from 486 nm [control: shown as (b) in FIG. 6A] to 505 nm [FIG. 6A, (a)], which should not occur with irregular aggregates. The point of maximal spectral difference between the polymer-containing solution and a dye-only solution was at 528 nm (FIG. 6B), which is shorter than that of amyloid fibrils (541 nm; Klunk, W. E. et al., (1999) Anal. Biochem., 266, 66-76). Therefore, CR-50 epitope fragments formed a string-like, soluble polymer with a regular structure, but that was different than amyloid fibrils.

### Example 7

Circular Dichroism (CD) Spectroscopy

To analyze the secondary structure of the CR-50 epitope fragment, circular dichroism (CD) spectroscopy was performed. CD spectra were measured with a Jasco spectropolarimeter, model J-725 equipped with a computer-controlled water bath to control the temperature at 20° C. The results were expressed as the mean residue ellipticity [θ]. CD spectra were measured at a protein concentration of 5 μM. The spectropolarimeter was purged with nitrogen gas, and scanned from 195 nm to 250 nm. Ten measurements were averaged to obtain each spectrum. The secondary structure contents were estimated from the ellipticity value (Greenfield, N. J. & Fasman, G. D., (1969) Biochemistry, 8, 4108-4116; Chen, Y. -H. et al., (1972) Biochemistry, 11, 4120-4131; Brahms, S. & Brahms, J., (1980) J. Mol. Biol., 138, 149-178; Greenfield, N. J. (1996) Anal. Biochem. 235, 1-10).

Under physiological conditions (0.15 M NaCl, pH 7.4), the CD spectrum of the CR-50 epitope fragment exhibited a broad negative minimum at 207 nm (0.15 M in FIG. 6C). From this result, the relative proportion of α-helix and β-sheet in the CR-50 epitope fragment was estimated to be about 20% and 40%, respectively (Greenfield, N. J. & Fasman, G. D., (1969) Biochemistry, 8, 4108-4116; Chen, Y. -H. et al., (1972) Biochemistry, 11, 4120-4131; Brahms, S. & Brahms, J., (1980) J. Mol. Biol., 138, 149-178).

### Example 8

Effect of Ionic Strength on Polymer Formation

To study the mechanism of polymer formation, the effect of ionic strength on polymer formation was examined. As salt (NaCl) concentration increased, the polymer gradually dissociated into monomers, indicating that this polymer is formed by electrostatic interaction (FIGS. 5A-5D). Finally, at 1.0 M NaCl, all fragments were present as monomers (FIG. 5D). This transition from polymer to monomer was reversible depending on the salt concentration. The CD spectral analysis indicated that, when the salt concentration increased, the negative minimum point shifted to a longer wave length and the ellipticity amplitude decreased (FIG. 6C). These results mean that the β-sheet content increased and α-helix content decreased. At 1.0 M NaCl, where the polymers completely dissociate into monomers (FIG. 5D), the negative minimum became 218 nm, indicating that almost all regular structure is β-sheet (FIG. 6C). Taken together, these results suggest that α-helix regions mainly contribute to the polymer formation.

The data presented here indicate that Reelin molecules assemble together to form a huge protein complex both in vitro and in vivo. As shown in FIG. 2A and as previously reported in Ogawa, M. et al., (1995) Neuron, 14, 899-912; deBerbeyck, V. et al., (1997) Mol. Brain Res., 50, 85-90; Miyata, T. et al., (1997) J. Neurosci., 17, 3599-3609; and Nakajima, K. et al., (1997) Proc. Natl. Acad. Sci. USA, 94, 8196-8201, some of the secreted Reelin is anchored to the surface of the Reelin-producing cells by an unknown mechanism. When Reelin protein molecules on living cells are immunostained with antibodies, they are labeled as many spots on the cell membrane, which may reflect the matrix of Reelin assembly. In addition, the Reelin protein molecules secreted from cultured cells were also found to form the homophilic complex in the medium. Thus, Reelin is thought to assemble irrespective of whether it is anchored to the cell surface or secreted into the extracellular space. When Reelin receptor-expressing cells migrate and interact with Reelin, the homophilic Reelin complex may function as “multivalent ligands” and induce receptor polymerization, resulting in transphosphorylation of intracellular Dab1 proteins by recruiting a protein kinase. This receptor polymerization may have been observed as “dots” that are co-localized with Reelin when embryonic brains were double-stained with anti-CNR protein and CR-50 antibodies (Senzaki, K. et al., (1999) Cell, 99, 635-647).

The assembly of Reelin protein molecules is thought to be accomplished by electrostatic interaction via the CR-50 epitope region, which is localized near the N-terminus of Reelin. CR-50 antibody appears to recognize a three-dimensional structure because it is very sensitive to various treatments such as fixation and ethanol treatment. This epitope region is essential for Reelin functions, because CR-50 antibody inhibits laminar pattern formation in cortical re-aggregation cultures (Ogawa, M. et al., (1995) Neuron, 14, 899-912), Purkinje cell alignment in cerebellar explant cultures (Miyata, T. et al., (1997) J. Neurosci., 17, 3599-3609), and an axonal branching pattern in the entorhino-hippocampal pathway in vitro (DelRio, J. Q. et al., (1997) Nature, 385, 70-74) and disrupts hippocampal development in vivo (Nakajima, K. et al., (1997) Proc. Natl. Acad. Sci. USA, 94, 8196-8201).

The results presented here indicate that the assembly of secreted Reelin molecules is inhibited by CR-50 antibody even at 20 μg/ml. Because Reelin function is inhibited by CR-50 antibody from around this concentration, it is highly likely that the Reelin assembly is perturbed when its function is blocked. Recently, lipoprotein receptors (VLDLR and ApoER2) and CNR family members were reported to bind to the full-length Reelin (D'Arcangelo et al., (1999) Neuron 24, 471-479; Hiesberger, T. et al., (1999) Neuron, 24, 481-489; Senzaki, K. et al., (1999) Cell, 99, 635-647). However, VLDLR and ApoER2 do not bind to a recombinant N-terminal portion of Reelin, which includes the CR-50 epitope region, and the CNR proteins bind to the subrepeat B of the first Reelin repeat, which is located downstream of the CR-50 epitope region (Senzaki, K. et al., (1999) Cell, 99, 635-647). These results indicate that the binding sites for all three are located outside of the CR-50 epitope region on Reelin. Further analysis, however, revealed that CR-50 antibody inhibited Reelin binding to VLDLR-expressing 293T cells (D'Arcangelo et al., (1999) Neuron 24, 471-479) and to the extracellular domain of CNR1 protein (Senzaki, K. et al., (1999) Cell, 99, 635-647). One possible interpretation of these results is that CR-50 binding to the N-terminus may inhibit the receptor-Reelin interaction indirectly by inducing structural change or steric hindrance to the binding sites on Reelin. Alternatively, or additionally, CR-50 antibody may decrease the number of Reelin protein molecules bound to the receptors by interfering with the huge homophilic complex formation of the ligand (Reelin) itself. Because the assembly of Reelin is inhibited by CR-50 antibody, each receptor may have bound to a monomer rather than to a huge complex of Reelin, which should have been difficult to be distinguished from the direct inhibition of the receptor-ligand interaction. Interestingly, the assembly of Reelin that was secreted from cerebellar granule cells was inhibited even at 20 μg/ml antibody (FIG. 3F). In contrast, Reelin binding to the lipoprotein receptor-expressing cells was reduced to 36% and 16% at 115 and 300 μg/ml CR-50 antibody, respectively (D'Arcangelo et al., (1999) Neuron 24, 471-479). The above-mentioned cell adhesion assay using full-length Reelin showed a substantially similar level of inhibition by this antibody (FIG. 3E; reduced to 44% and 32% at 50 and 200 μg/ml CR-50 antibody). Taking into account that the adhesion assay using cultured cells gives high background in general (FIGS. 2D-2G, 4B and 4C), these data suggest that CR-50 antibody may neutralize Reelin function (at least in part) by inhibiting the homophilic assembly formation of the Reelin protein itself. Accordingly, it is important to clarify whether the binding of Reelin monomers to the receptors could initiate appropriate signaling to control neuronal migration.

Purified CR-50 epitope fragments assemble spontaneously to form a soluble homopolymer with a regular repeated structure. It has α-helix+β-sheet structure, of which the α-helix region contributes mainly to the polymer formation. The secondary structure prediction by the method of Chou and Fasman (Chou, P. Y. & Fasman, G. D. (1978) Adv. Enzymol., 47, 45-148) suggested that the epitope should have a long α-helix domain at its C-terminus and a short α-helix domain in the middle. In fact, these domains contain many (about 30%) charged amino acids, which appear to be responsible for the polymer formation. One possibility is that these two domains function as hinges to assemble monomers to make the linear, string-like structure of the polymer. A pH-titration experiment showed that the CR-50 epitope fragments take the original α-helix+β-sheet structure only under physiological conditions (data not shown). We also found that the electrostatic interaction between CR-50 epitope fragments depends on the surface charge density of the CR-50 epitope.

One interesting finding in the gel filtration analysis was that under physiological conditions there was only a huge polymer peak, but no monomer peak (FIG. 5A). However, Western blotting of the full-length Reelin showed a broad range of bands from the monomer to the top of a native gel (FIGS. 1B-1D), which indicates that there are various polymers composed of different numbers of monomers. This suggests that the CR-50 epitope region itself assembles spontaneously, which then may drive full-length polymer formation. In the case of the full-length molecules, steric hindrance or some other mechanisms may keep them at various stages of polymerization.

The present invention provides the first elucidation that the assembly of Reelin protein molecules in the developing brain of a vertebrate is mediated by the epitope region against its function-blocking antibody, CR-50, which clearly disrupts this interaction. Reelin is suggested to function as a large, homophilic complex in vivo to control neuronal migration in neurodevelopment. The polypeptide of the present invention may be used to examine the status of this complex formation.

In addition to clarification of Reelin protein functions in vivo as described above, the present invention can also be utilized for appropriate diagnosis and treatment of brain disorders resulting from an abnormal reelin gene and abnormally positioned neurons, such as lissencephaly, polymicrogyria, and ectopic gray matter, by stimulating the assembly of Reelin protein molecules using the polypeptide of the present invention or a polynucleotide encoding it.

Because the polypeptide of the present invention contains neither F-spondin domain nor repeat site, it is characterized by smaller molecular size and a greater addition advantage, and can be used only for the intended complex formation. The polypeptide of the present invention has advantageous features presented below.

First, when administering a substance to an individual (patient) for the purpose of treatment, since regardless of administration route, this involves artificially administering a substance that the individual originally lacks, it results in the introduction of a foreign substance to that individual. Administration of this foreign substance may induce not only the intended main effect but also side effects. In the present invention, by identifying the epitope region as an essential minimal polypeptide useful in treatment, when administering a polynucleotide or polypeptide covering only the epitope region, since portions unnecessary for treatment are not included, foreign substance-induced side effects can be minimized.

Secondly, when attention is directed to only the epitope region which is a minimal region necessary for treatment, the preparation cost of a polynucleotide encoding the region and a polypeptide corresponding to the region can be minimized.

All publications, patents and patent applications cited herein are incorporated by reference in their entirety.

What is claimed is:

1. A CR-50 epitope region polypeptide of Reelin protein, which comprises a CR-50 antibody recognition site of Reelin protein, but comprises neither a F-spondin domain nor a repeat site.

2. The CR-50 epitope region polypeptide of Reelin protein according to claim 1, which is derived from a mouse.

3. A polypeptide selected from the group consisting of:
(a) a polypeptide comprising the amino acid sequence shown in SEQ ID NO: 2; and
(b) a polypeptide capable of binding to CR-50 antibody, which comprises deletion, substitution or addition of one or more amino acids in the amino acid sequence shown in SEQ ID NO: 2.

4. A polynucleotide encoding the polypeptide according to any one of claims 1 to 3.

5. A polynucleotide selected from the group consisting of:
(a) a polynucleotide comprising the nucleotide sequence shown in SEQ ID NO: 1;
(b) a polynucleotide encoding a polypeptide capable of binding to CR-50 antibody, which comprises deletion, substitution or addition of one or more nucleotides in the nucleotide sequence shown in SEQ ID NO: 1; and
(c) a polynucleotide comprising a degenerate nucleotide sequence of polynucleotide (a) or (b).

6. An expression vector comprising the polynucleotide according to claim 4 or 5.

7. A host cell transfected with the expression vector according to claim 6.

8. A method for producing the polypeptide according to any one of claims 1 to 3, which comprises culturing the host cell according to claim 7.

9. A method for stimulating the assembly of Reelin protein molecules, which comprises adding the polypeptide according to any one of claims 1 to 3 or the polynucleotide according to claim 4 or 5.

10. A composition for stimulating the assembly of Reelin protein molecules, which comprises the polypeptide according to any one of claims 1 to 3 or the polynucleotide according to claim 4 or 5.

11. A pharmaceutical composition for diagnosis and/or treatment of diseases resulting from abnormally positioned neurons, which comprises the polypeptide according to any one of claims 1 to 3 or the polynucleotide according to claim 4 or 5.
